(19) United States
(12) Patent Application Publication
(10) Pub. No.: US 2026/0077193 A1
(43) Pub. Date: Mar. 19, 2026

(54) NON-INVASIVE PERIPHERAL NERVE STIMULATION SYSTEM AND METHOD FOR TREATING ANXIETY AND INSOMNIA

(71) Applicant: Roga Life Canada, Inc., Toronto (CA)

(72) Inventors: Ami Lebendiker, San Diego, CA (US); Alison Smith, Toronto (CA); Brian Cooper, San Francisco, CA (US)

(21) Appl. No.: 19/012,481

(22) Filed: Jan. 7, 2025

Related U.S. Application Data
(60) Provisional application No. 63/694,235, filed on Sep. 13, 2024.

(51) Int. Cl. A61N 1/36; A61N 1/02; A61N 1/04; G16H 10/20; G16H 20/70; G16H 40/63

(52) U.S. Cl. CPC A61N 1/36025; A61N 1/025; A61N 1/0456; A61N 1/0496; A61N 1/36034; G16H 10/20; G16H 20/70; G16H 40/63

(57) ABSTRACT

A Peripheral Nerve Stimulation (PNS) system and method are disclosed. The system includes a headset with ear hooks and electrodes for placement on the mastoid processes. A stimulation device connected to the headset via lead wires delivers a pulsed electrical current to the electrodes. The device communicates with a computing device equipped with a mobile application that controls the stimulation characteristics. The mobile application allows users to adjust settings, monitor progress, and access treatment sessions. The system also includes snap ports for electrode attachment, self-adhesive hydrogel electrodes, and current control mechanisms using a digital potentiometer and waveform generator. The method includes pre- and post-session anxiety rating, personalized treatment based on user inputs, and tracking of physiological biomarkers. The application supports multiple operating systems, secure cloud data storage, and enables data sharing with healthcare providers. The invention provides a comprehensive, customizable, safe solution for managing anxiety and insomnia.

### FIELD OF INVENTION

The instant application claims priority to U.S. provisional application 63/694,235, filed on Sep. 13, 2024, presently pending. The contents of each application are hereby incorporated by reference.

The present invention relates generally to medical devices and methods for treating anxiety and insomnia. More specifically, the invention pertains to a non-invasive peripheral nerve stimulation (PNS) system and method for treating anxiety and insomnia.

### BACKGROUND

In the field of non-invasive medical devices for treating anxiety and insomnia, existing methods typically involve non-pharmacological approaches, such as cognitive behavioral therapy (CBT), mindfulness techniques, and various relaxation methods. These methods often rely on behavioral interventions rather than direct physiological stimulation. For instance, traditional therapies may include relaxation exercises, biofeedback techniques, or guided meditation sessions aimed at reducing stress and promoting sleep.

Some devices and systems involve the use of electrical stimulation for therapeutic purposes, though not specifically targeted at peripheral nerve stimulation for anxiety and insomnia. For example, transcutaneous electrical nerve stimulation (TENS) devices are used for pain management and muscle stimulation, typically applied through adhesive electrodes placed on the skin.

Existing non-invasive peripheral nerve stimulation (NI-PNS) devices for the treatment of anxiety and/or insomnia are not designed to be compact and portable. The current market offerings are too large to fit into a pocket, limiting their use to specific locations where the device can be comfortably accommodated. Also, the available NI-PNS devices are not scalable due to their high manufacturing costs. These devices that are produced are expensive to manufacture, resulting in high product costs for patients, making them less accessible to a broader audience. Further, some of these NI-PNS devices require the use of electrode gel, which is necessary for the proper functioning of dry electrodes. There are NI-PNS devices with dry clip-on earlobe electrodes or electrodes embedded within large earphones. The application of this gel is often messy and inconvenient, leading to potential staining of clothing and discomfort for the patient. Some of the existing NI-PNS devices are not designed with discretion in mind. The conspicuous nature of the devices, especially those with wired ear clip electrodes or large earphones, makes them impractical for use in public settings. This reduces the likelihood of regular usage due to social discomfort.

Current NI-PNS devices do not come with integrated mobile applications that could enhance the user experience. There is no provision for patients to assess their anxiety levels before or during treatment, nor is there any analytical tracking of treatment progress over time. Also, some NI-PNS devices, particularly those with large earphones, compromise the user's ability to hear their surroundings. This makes it impractical to use these devices in environments where situational awareness is crucial, such as at work or in public spaces.

In recent years, advancements in wearable technology and mobile applications have enabled the integration of physiological monitoring and therapeutic interventions. Wearable devices with biofeedback capabilities allow users to monitor physiological indicators like heart rate variability, skin conductance, and temperature, which can be indicators of stress and anxiety levels. Mobile applications have been developed to provide users with interactive treatment sessions, customizable therapeutic content, and progress tracking functionalities.

Despite these advancements, there remains a need for improved systems that specifically target anxiety and insomnia through non-invasive peripheral nerve stimulation. The present invention addresses this need by providing an integrated approach aimed at enhancing user experience, treatment efficacy, and accessibility compared to existing therapeutic solutions.

Therefore, while various technologies and methods exist for managing anxiety and insomnia through non-invasive means, the present invention represents a novel integration of peripheral nerve stimulation with a user-friendly design and advanced mobile application functionalities tailored for these specific therapeutic purposes.

### SUMMARY

The present invention relates to a non-invasive peripheral nerve stimulation (PNS) system, method, and device for the treatment of anxiety and insomnia, providing an ergonomic, portable, and user-friendly solution that integrates with mobile technology to offer personalized and interactive treatment sessions.

The present invention relates, in one embodiment, to a system. The system includes a headset with two ergonomically designed ear hooks that fit around the ears of a user. Each ear hook is equipped with an adhesive electrode that is placed on the skin of the user's mastoid processes. The system includes a stimulation device that delivers a pulsed electrical current to the electrodes via lead wires. This stimulation device is connected to a computing device, such as a smartphone, with a mobile application installed. The mobile application allows users to control various output stimulation characteristics, monitor treatment progress, and access interactive treatment sessions, thereby offering a customizable and user-friendly treatment experience.

The present invention relates, in another embodiment, to a method. This method is for treating anxiety and insomnia through a mobile application in communication with a concealable, non-invasive stimulation device. The method involves presenting a user interface on the mobile application where users can select between two modes of interactive treatment sessions: pre-recorded content mode and dynamically generated meditation mode. Upon selecting the pre-recorded content mode, the user inputs a pre-session anxiety rating, initiates playback of the content, and inputs a post-session anxiety rating after the session. For the dynamically generated meditation mode, users provide verbal or written input parameters, configure the session based on these inputs, and similarly input anxiety ratings before and after the session. This method ensures a tailored treatment approach, enhancing the effectiveness of the therapy.

The present invention relates, in another embodiment, to an apparatus. A concealable peripheral nerve stimulation (PNS) apparatus specifically for the treatment of anxiety and insomnia. This apparatus comprises a headset with two ear hooks that fit comfortably around the ears, each connected to an adhesive electrode placed on the mastoid processes. It further includes a stimulation device that is designed to generate various output stimulation characteristics, delivering a pulsed electrical current through the lead wires. Users can control these stimulation characteristics to tailor the treatment to their specific needs, ensuring effective management of anxiety and insomnia.

The present invention addresses the technical problems present in the conventional technologies by offering a NI-PNS device that has various features. It is small enough to fit into a pocket, allowing patients to carry and use the device anywhere. It is designed to be cost-effective in production, resulting in a lower price point for patients. It utilizes adhesive, disposable electrodes that do not require the use of conductive gel, thereby avoiding the mess and inconvenience associated with gel application. It is also designed to be unobtrusive, enabling patients to use the device in public without drawing attention. It includes an integrated comprehensive mobile application for monitoring anxiety levels, tracking treatment progress, and providing supportive content to enhance user engagement. It does not compromise the user's ability to hear their surroundings, ensuring usability in various environments.

One of the objects of the present invention is to offer a method of treatment that allows for personalized therapy sessions based on user inputs and real-time monitoring of anxiety levels. One of the technical advantages of the present invention is providing integration with a secure cloud server for data storage and synchronization ensuring that user data is protected and easily accessible for tracking treatment progress and sharing with healthcare providers.

### BRIEF DESCRIPTION OF THE DRAWINGS

The invention will be more readily understood from the following detailed description of the preferred embodiments, taken in conjunction with the accompanying drawings, in which like reference numerals designate like structural elements, and wherein:

FIG. 1 illustrates an overall design of the Peripheral Nerve Stimulation (PNS) apparatus, in accordance with one embodiment of the present invention.

FIG. 2 illustrates a detailed view of the ear hooks of the Peripheral Nerve Stimulation (PNS) apparatus, in accordance with one embodiment of the present invention.

FIG. 3 shows the placement of the headset on a user and/or patient, in accordance with one embodiment of the present invention.

FIG. 4 shows an exploded view of the stimulation device, in accordance with one embodiment of the present invention.

FIG. 5 shows a user interface of an accompanying mobile application displaying the home screen and anxiety intensity survey within the mobile application, in accordance with one embodiment of the present invention.

FIG. 6 is a view of the screens of the mobile application before and after an interactive treatment session, in accordance with one embodiment of the present invention.

FIG. 7 is a view of the screen of the mobile application shown to the user after a pre-recorded content mode of the interactive treatment session, in accordance with one embodiment of the present invention.

FIG. 8 is a view of the screen from a mobile application designed to display a user's anxiety intensity score, in accordance with one embodiment of the present invention.

FIG. 9 is a view of the screen of the mobile application interface that facilitates the creation of interactive treatment sessions, including a dynamically generated meditation mode tailored to the user's needs, in accordance with one embodiment of the present invention.

FIG. 10(a) is a view of the screen of the mobile application where the user can select the pre-recorded content mode, in accordance with one embodiment of the present invention.

FIG. 10(b) is a view of the screen of the mobile application where the user can select the intensity level for the stimulation, in accordance with one embodiment of the present invention.

FIG. 11 is a view of the screen of the mobile application containing the user profile and anxiety intensity statistics, in accordance with one embodiment of the present invention.

FIG. 12 is a block diagram of the Peripheral Nerve Stimulation (PNS) system, in accordance with one embodiment of the present invention.

FIG. 13 is a flowchart showing stimulation device operation steps of the Peripheral Nerve Stimulation (PNS) apparatus, as shown in FIG. 1, in accordance with one embodiment of the present invention.

FIG. 14 is a flowchart showing the operational steps required to complete the interactive treatment sessions in the pre-recorded content mode or a dynamically generated meditation mode, in accordance with one embodiment of the present invention.

FIG. 15 is a flow diagram of the configuration of the stimulation device, in accordance with one embodiment of the present invention.

### DETAILED DESCRIPTION OF THE INVENTION

Embodiments of the invention are discussed below with reference to FIGS. 1-15. However, those skilled in the art will readily appreciate that the detailed description given herein with respect to these figures is for explanatory purposes as the invention extends beyond these limited embodiments.

The present invention is a non-invasive Peripheral Nerve Stimulation (NI-PNS) system designed for the treatment of anxiety and/or insomnia. The invention comprises a miniaturized stimulation device that is small enough to fit in a pocket and, when worn, is completely concealable, thus promoting its use in public spaces.

The present invention is easy to use, quick to put on (in less than 30 seconds) and hidden from view by others. Users and/or patients suffering from anxiety can easily self-administer treatment as needed and carry the device in their pocket or bag. During treatment, patients snap disposable, adhesive electrodes onto the headset ear hooks and place the electrodes on the skin of the mastoid processes behind each ear. The device emits a low-level, pulsed, electrical square wave current that feels like a gentle tingling on the skin. The device is typically worn for 20-60 minutes per day for up to 6 weeks to reduce symptoms of anxiety and/or insomnia, and then 2-3 times per week thereafter to maintain results.

In addition to the stimulation device, the users and/or patients have access to a mobile application that offers several functionalities such as creation of a user profile, completion of a short validated psychological questionnaire to obtain an anxiety score, rating of anxiety intensity before and after each treatment session, tracking of anxiety intensity over time, writing journal entries, listening to pre-recorded guided meditations, creation of dynamically generated guided meditations based on personal inputs, setting a silent meditation timer and treatment reminders. This invention may reduce a patient's need for medication or cognitive behavioral therapy and may assist medical centers with long patient waitlists.

FIG. 1 illustrates an overall design of the Peripheral Nerve Stimulation (PNS) apparatus for the treatment of anxiety and/or insomnia, in accordance with one embodiment of the present invention. The apparatus includes a headset with two flexible ear hooks 11 that ergonomically fit around the ears of the user. Each ear hook 11 features a flexible drop wire 12 and a snap port or snap connector 13 for attaching adhesive electrodes to the headset and the skin of the mastoid processes. In an example, the snap port 13 can be a 3.9 mm snap port. Electrodes used with the device are self-adhesive, conductive hydrogel disposable electrodes.

The headset is connected via a flexible lead wire 14 that terminates in a jack 15. This jack 15 plugs into a corresponding port 16 on the stimulation device 17. In an example, the jack 15 can be a 2.5 mm jack and the port 16 can be a 2.5 mm port. The headset ear hooks 11 are designed to follow the contour of the ear, made of a soft, flexible material for ease of use, and allow for precise electrode placement on the mastoid processes. The snap port 13 secures disposable, adhesive electrodes, which use a solid electroconductive gel, eliminating the need for messy electroconductive liquids. The lead wire 14 resembles a typical wired earphone cable, allowing the headset to be used discreetly in public spaces. The miniature stimulation device 17, which is smaller than 5 cm×2.2 cm×1.5 cm and weighs less than ˜200 g, is designed for portability and ease of use, fitting comfortably in a pocket. It receives power from a computing device and does not contain a battery, simplifying shipping and enhancing portability. For example, the computing device may be a smartphone, a handheld device, a tablet, and the like. The overall design promotes the use of the NI-PNS apparatus by making it discreet, portable, and easy to use, thereby encouraging treatment compliance.

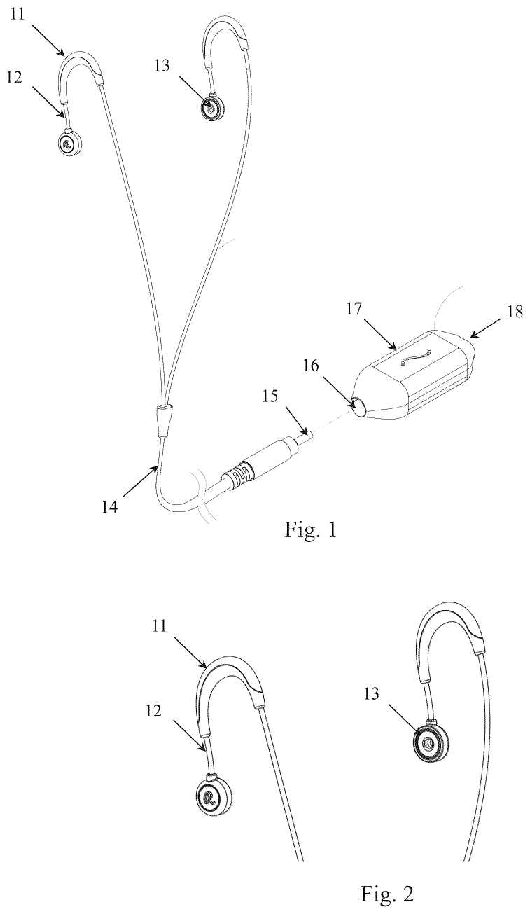

FIG. 2 illustrates a detailed view of the ear hooks of the Peripheral Nerve Stimulation (PNS) apparatus, in accordance with one embodiment of the present invention. This figure highlights the components that facilitate the attachment and positioning of the adhesive electrodes. Each ear hook 11 is designed to ergonomically fit around the user's ear, ensuring comfort and secure placement during use. The ear hooks are made from a soft, flexible material, which allows them to conform to the contours of the user's ears, providing an unobtrusive and comfortable fit. Attached to each ear hook 11 is a flexible drop wire 12. The drop wire 12 is crucial for positioning the electrodes correctly on the mastoid processes, which are located behind the user's ears. The flexibility of the drop wire 12 and length ensures precise placement of the electrodes, which is essential for effective treatment. At the end of each drop wire 12, there is a snap port 13. The snap port 13 is used to attach the adhesive electrodes to the headset and subsequently to the skin. In an example, the snap port can be a 3.9 mm snap port. The snap port 13 secures disposable, adhesive electrodes that contain a solid electroconductive gel. This design choice eliminates the need for messy electroconductive liquids, making the device easy and clean to use. The detailed illustration in FIG. 2 focuses on these components, showing how the ear hooks 11, flexible drop wires 12, and snap ports 13 work together to ensure the electrodes are correctly positioned and securely attached. This setup promotes ease of use, comfort, and discretion, encouraging regular use of the device for the treatment of anxiety and/or insomnia.

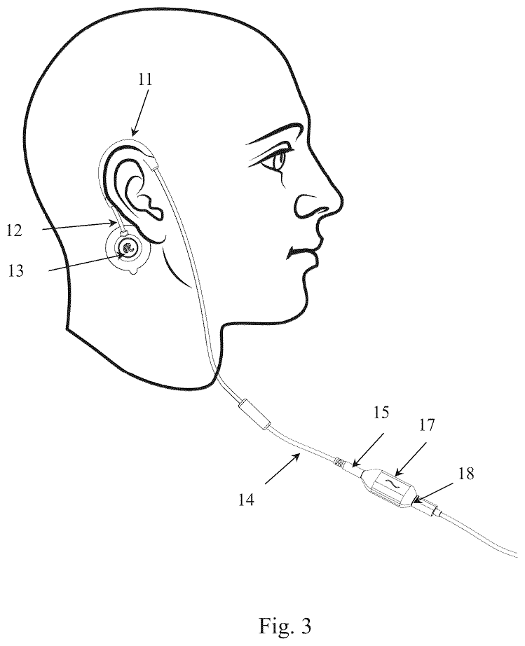

FIG. 3 shows the placement of the headset on a user and/or patient, in accordance with one embodiment of the present invention. This figure is specifically illustrating the right side of the head. The ear hook 11 of the headset follows the contour of the right ear, ensuring an ergonomic and secure fit. While the figure depicts the right ear, a similar ear hook is placed on the left ear, though it is not shown. Attached to the ear hook 11 is a drop wire 12, which contains a snap connector 13 designed to secure an adhesive electrode in place. The adhesive electrode is then adhered to the skin of the mastoid process, located behind the ear. The stimulation device 17, connected to the headset via a lead wire 14, provides a low-level, pulsed electrical current to the electrodes. This current is delivered through a jack 15 that plugs into a port 16 on the stimulation device. In an example, the jack 15 can be a 2.5 mm jack and the port 16 can be a 2.5 mm port. The entire setup, including the stimulation device 17 and the connection 18 to the power source, is designed to be discreet and portable, promoting ease of use and treatment compliance.

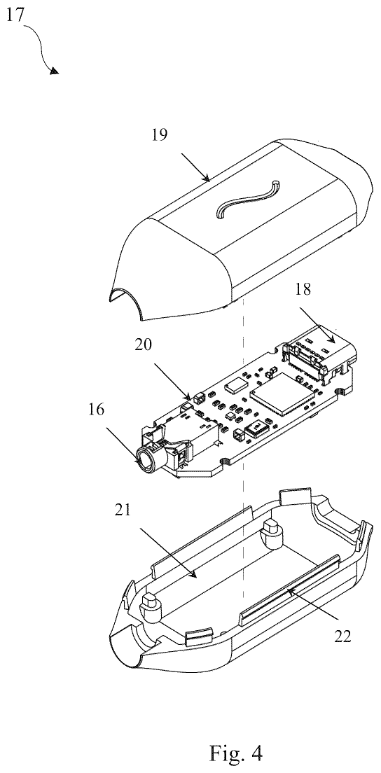

FIG. 4 shows an exploded view of a stimulation device, in accordance with one embodiment of the present invention. The stimulation device 17 comprises an upper plastic enclosure 19 and a lower plastic enclosure 21, both designed to protect and house the internal circuitry. These enclosures snap together using a snap closure 22, ensuring secure assembly and protection of the internal components. The circuit board 20, located within the enclosures, is the core of the stimulation device 17, containing essential electronic components. It features a jack port 16 for connecting a headset lead wire jack 15, facilitating the use of external accessories necessary for the device's functionality. In an example, the jack 16 can be a 2.5 mm jack. Additionally, the circuit board 20 includes a USB-A port 18 that allows for connection to a USB-A power adapter of the computing device, providing a means to charge or power the device through a standard USB connection. This assembly ensures that stimulation device 17 is both functional and well-protected from external damage.

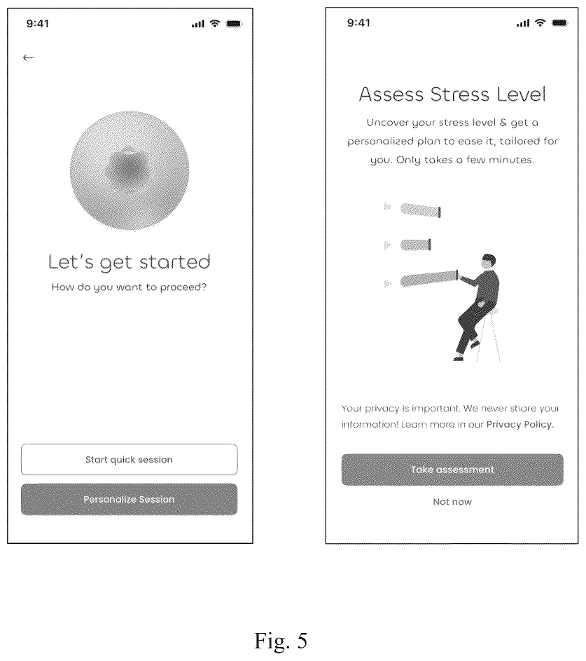

FIG. 5 shows a user interface of an accompanying mobile application displaying the home screen and anxiety intensity survey within the mobile application, in accordance with one embodiment of the present invention. This figure presents a user interface of a mobile application designed to accompany a non-invasive peripheral nerve stimulation (PNS) system. The mobile application controls a plurality of output stimulation characteristics of the stimulation device, wherein the mobile application allows the user to adjust the output stimulation characteristics, monitor treatment progress, and access a plurality of interactive treatment sessions. The output stimulation characteristics of the stimulation device includes intensity level of the pulsed electrical current, stimulation frequency, treatment duration, and operation mode. The pulsed electrical current is delivered in a low-level pulsed electrical square waveform. The figure displays two screens: the home screen and the anxiety intensity survey screen. On the home screen, the users are prompted to begin their session with options to either start a quick session or personalize their session. The anxiety intensity survey screen invites users to assess their stress levels through a series of questions from a validated psychological questionnaire tailored to measure anxiety intensity on a scale of 100.

This mobile application allows the users to create profiles, engage with the validated psychological questionnaire to assess anxiety symptoms, rate their anxiety intensity before and after interactive treatment sessions, and track their anxiety over time. Furthermore, it enables users to share their data with healthcare providers. The mobile application measures a plurality of physiological biomarkers associated with anxiety intensity, wherein the physiological biomarkers are selected from heart rate variability, heart rate, skin impedance, skin conductance, blood oxygenation, and skin temperature, to provide personalized treatment recommendations. The mobile application is available on multiple operating systems and requires users to create a password-protected account for secure access. The anxiety rating is based on Generalized Anxiety Disorder (GAD) parameters, including but not limited to nervousness, lack of control, excessive worry, tension, restlessness, irritability, and fear. The mobile application thus serves as a comprehensive tool for the users and/or patients to monitor and manage their anxiety symptoms effectively.

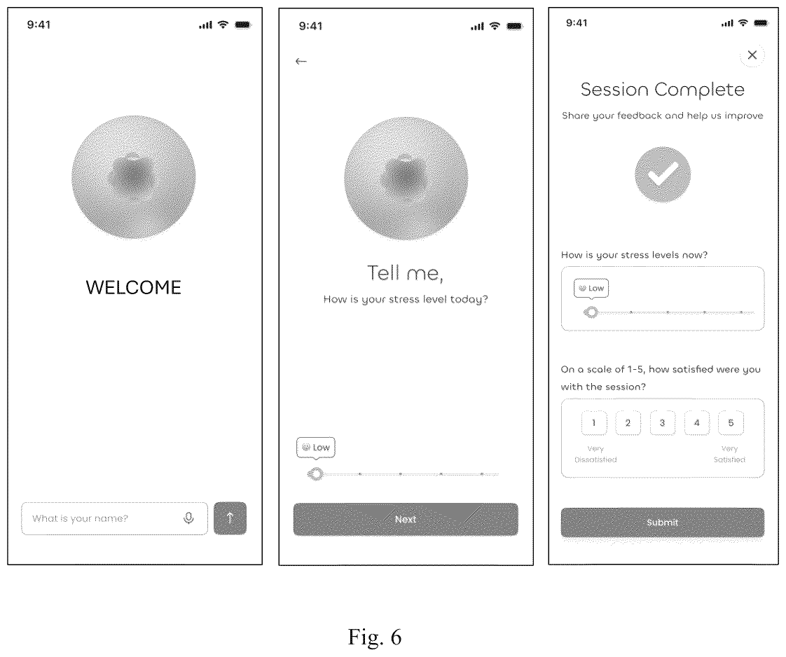

FIG. 6 is a view of the screens of the mobile application before and after an interactive treatment session, in accordance with one embodiment of the present invention. The first screen welcomes the user with a prompt to enter their name, initiating a personalized experience. The subsequent screen asks the user to report their current stress level using a Likert scale from 0 to 10. This pre-session assessment helps establish a baseline for the user's anxiety intensity, which will be tracked and compared to post-session results.

Following the interactive treatment session, which includes either a pre-recorded content mode or a dynamically generated meditation mode, the mobile application presents a screen to gather post-session feedback. The user is again asked to rate their current stress level on the same Likert scale, enabling the application to measure any changes in anxiety intensity. Additionally, the screen includes a satisfaction survey, where users can rate their session experience on a scale from 1 to 5. This dual assessment approach, both before and after the session, allows users to monitor the effectiveness of the treatment and provides valuable feedback for improving the mobile application's content and user experience.

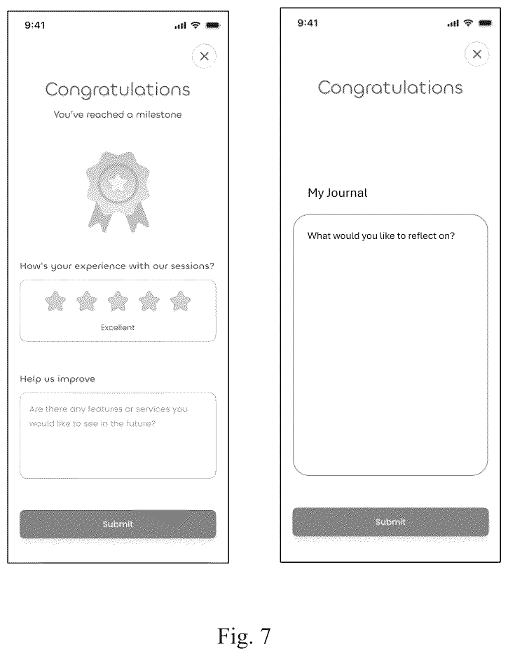

FIG. 7 is a view of the screen of the mobile application shown to the user after a pre-recorded content mode of the interactive treatment session, in accordance with one embodiment of the present invention. This figure illustrates two screens of a mobile application interface displayed to the user upon reaching a milestone in an interactive treatment session utilizing the pre-recorded content mode. The left screen shows a “Congratulations” message indicating the achievement of a milestone. Below the congratulatory message, the user is prompted to rate their experience with the sessions using a five-star rating system. This is accompanied by a text field labeled “Help us improve,” where users can submit their suggestions or feedback for additional features or services they would like to see in the future.

The right screen is labeled “My Journal” and features a text box prompting the user with “What would you like to reflect on?” Here, users can input their journal entries, allowing them to reflect on their experiences and submit the same. These screens are part of the mobile application's user interface designed to collect user feedback and personal reflections, which are integral components of enhancing user engagement and improving the therapeutic experience.

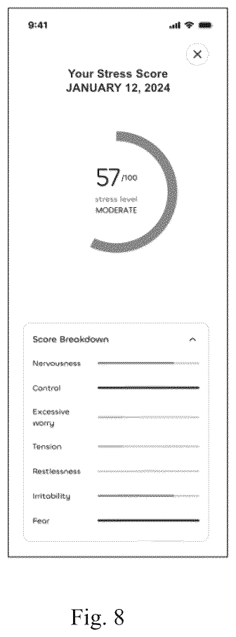

FIG. 8 is a view of the screen from a mobile application designed to display a user's anxiety intensity score over time, in accordance with one embodiment of the present invention. The anxiety score is recorded, and the same is displayed on the screen. For example, the screen displays an anxiety score of “57/100” with a corresponding stress level labeled “Moderate.” This score is calculated using the Generalized Anxiety Disorder 7 (GAD-7) scale and is converted to a percentage. This conversion involves taking the GAD-7 score, which ranges from 0 to 21, dividing it by 21, and multiplying the result by 100 to produce a percentage. For instance, a GAD-7 score of 15 would correspond to a 71% anxiety score. A “Score Breakdown” section is provided, detailing various GAD parameters contributing to the overall anxiety score. These parameters include Nervousness, Control, Excessive Worry, Tension, Restlessness, Irritability, and Fear, each represented with a corresponding bar indicating their individual scores.

The mobile application allows users to track their anxiety intensity over time, presenting data derived from validated psychological questionnaires such as the GAD-7 and Likert scales. The users are prompted to rate their anxiety levels before and after each interactive treatment session on a 10-point Likert scale. This data enables users to monitor changes in their anxiety levels across multiple treatment sessions and share this information with their healthcare providers. The integration of such metrics into the mobile application supports users in managing their anxiety more effectively while using the non-invasive peripheral nerve stimulation (PNS) system.

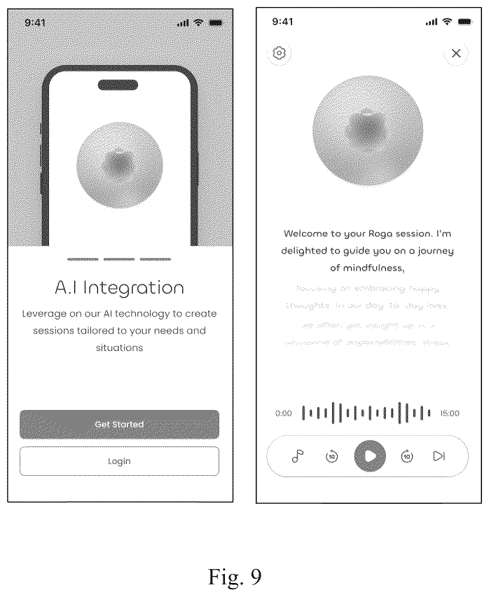

FIG. 9 is a view of the screen of the mobile application interface that facilitates the creation of interactive treatment sessions, including a dynamically generated meditation mode tailored to the user's needs, in accordance with one embodiment of the present invention. The left screen introduces the concept of “A.I. Integration,” emphasizing the mobile application's use of artificial intelligence to create personalized sessions based on the user inputs. The user can either begin the personalization process or log into their account. The right screen provides a welcome message for a meditation session, indicating that the user is about to embark on a mindfulness journey. The user interface includes an audio control bar at the bottom, featuring playback controls and a progress bar showing a session duration of 15 minutes. This screen suggests that users receive guidance through a narrated session designed to help them focus on positive thoughts and mindfulness. The personalization process allows users to input specific details about the causes of their anxiety, preferences for the narrator's voice (male or female), session duration, type of music, and the language of the guided meditation. These inputs enable the mobile application to generate highly customized meditation sessions that cater to the user's individual needs. This dynamic generation of sessions encourages continued use of the mobile application and enhances the therapeutic experience by providing tailored content that addresses the user's unique stressors and preferences.

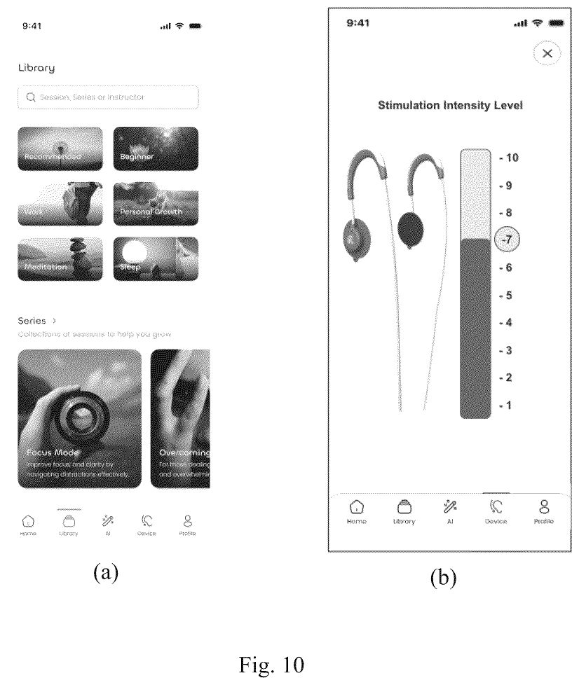

FIG. 10(a) is a view of the screen of the mobile application where the user can select a pre-recorded content mode or a dynamically generated meditation mode, in accordance with one embodiment of the present invention. This figure depicts the mobile application's main library screen, where users can select from pre-recorded content modes or dynamically generated meditation sessions. This screen allows users to access various guided meditation sessions, including those aimed at beginners, personal growth, work-related stress, meditation, and sleep.

FIG. 10(b) is a view of the screen of the mobile application where the user can select the intensity level for the stimulation, in accordance with one embodiment of the present invention, which shows the screen where users can control the intensity level of the stimulation therapy. This figure shows the screen where the users can control the intensity level of the stimulation therapy. The intensity can be adjusted between 5 μA and 2 mA, with levels ranging from 1 to 10. The duration of the treatment is also modifiable, ranging from 5 to 60 minutes, and the device automatically shuts off after 60 minutes. The device emits waveforms at preset frequencies of 0.5, 1.5, and 100 Hz.

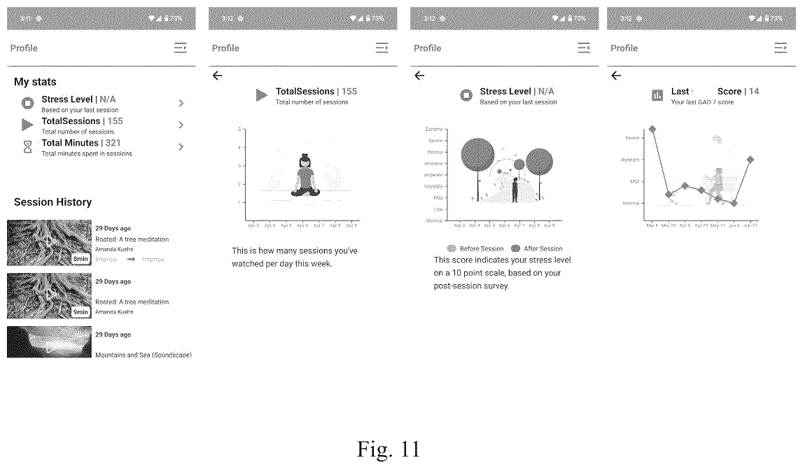

FIG. 11 is a view of the screen of the mobile application containing the user profile and anxiety intensity statistics in accordance with one embodiment of the present invention. This figure illustrates the user profile screen, displaying detailed analytics and statistics related to the user's treatment and anxiety management progress. The screen is divided into four main sections: the Profile Overview, the Total Sessions, the Stress Level, and the Last GAD7 Score. The profile overview section summarizes key metrics, including the user's stress level based on their last session, the total number of sessions completed, the total minutes spent in sessions, and the most recent GAD-7 score, a standard tool for measuring anxiety severity. Additionally, it includes a session history that lists past sessions with details such as title, duration, instructor, and intensity level. One section of the profile displays a graph showing the number of sessions the user has completed each day over the past week, helping users track their engagement with the mobile application. Another section shows the user's stress levels before and after sessions, based on a 10-point scale derived from post-session surveys. This visual representation helps users understand how their stress levels fluctuate and improve with treatment. The last section provides a detailed view of the user's latest GAD-7 score, explaining what the GAD-7 is and how it's used to measure anxiety symptoms. It also includes a graph showing changes in GAD-7 scores over time, offering insight into the user's long-term progress.

The mobile application ensures security by requiring users to create a password-protected account. It records detailed treatment data, including session date, start and end times, duration, intensity, frequency, and session history. The mobile application analyzes treatment progress using validated psychological questionnaires and displays the data through various graphs and statistics. It also provides push notifications for treatment reminders and allows users to download their encrypted and de-identified treatment data in a .csv file from a secure cloud server to share with their healthcare provider. The data is securely stored in AWS Cloud Servers. Additionally, the mobile application can operate as a software-as-a-service (SaaS) product, allowing payments from patients or healthcare providers.

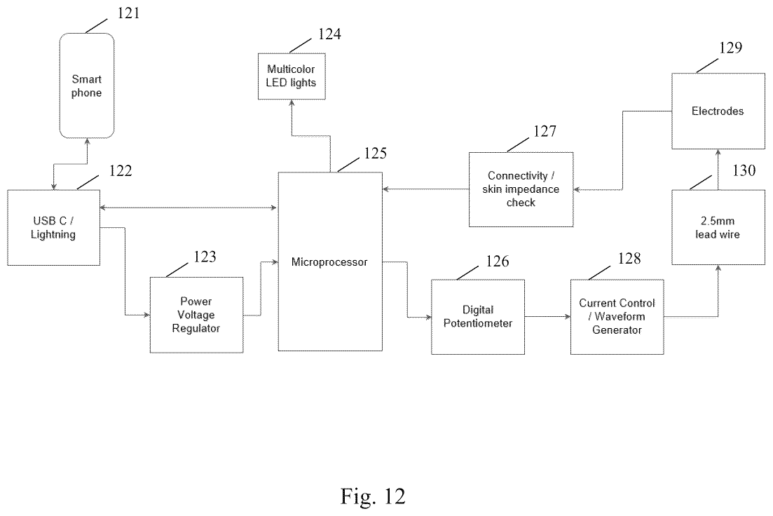

FIG. 12 is a block diagram of the Peripheral Nerve Stimulation (PNS) system, in accordance with one embodiment of the present invention. This figure highlights the interaction and functionality of each component within the system. The stimulation device operates in conjunction with a smartphone 121, which acts as its power source and control interface. The smartphone connects to the stimulation device via a USB-C or Lightning port 122, allowing it to supply power and facilitate communication between the mobile application and the stimulation device. Once connected, the power voltage regulator 123 steps down and stabilizes the incoming voltage from the smartphone to levels suitable for the PNS device's internal circuitry. The regulated power is then fed to the microprocessor 125, which serves as the central control unit of the system. The microprocessor orchestrates various functions, including the activation of multicolor LED lights 124 that indicate the device's operational status.

The microprocessor 125 also interfaces with the connectivity/skin impedance check module 127, which verifies that the electrodes 129 are properly attached to the user's skin. This check ensures that the stimulation is delivered effectively and safely. If the electrodes are not correctly positioned, the system can prompt the user to adjust them, preventing ineffective treatment or potential discomfort. The digital potentiometer 126 plays a crucial role in controlling the intensity of the electrical stimulation. It allows for fine-tuning of the current based on inputs from the mobile application, ensuring that the stimulus remains within safe and therapeutic limits. The current control and waveform generator 128 then creates the specific electrical waveforms needed for nerve stimulation. These waveforms have variable pulse widths, ranging from 5 milliseconds to 1 second, depending on the frequency settings provided by the application. This variability allows for customized treatment protocols tailored to individual patient needs.

The generated waveforms are transmitted through a lead wire 130 to the electrodes 129, which are attached to the user's skin. In an example, the lead wire 130 can be a 50 cm long lead wire. These electrodes deliver the precise electrical stimulation required for the therapy. To prevent any accidental over-stimulation, the system incorporates both hardware and software safety features. Physical resistors and IO pins within the integrated circuit limit the current flow to a maximum of 2 mA. Additionally, the software-controlled digital potentiometer 126 ensures that any surge in current does not exceed this threshold. For further safety, the stimulation device includes an integrated timer that automatically shuts off the power supply after 60 minutes of continuous operation. This feature prevents prolonged exposure to the therapy, which could potentially cause adverse effects. This figure details a sophisticated PNS system that integrates power management, safety checks, and customizable stimulation control, all regulated by a microprocessor and a mobile application. This design ensures that the therapy is both effective and safe, providing users with a reliable tool for managing their peripheral nerve conditions.

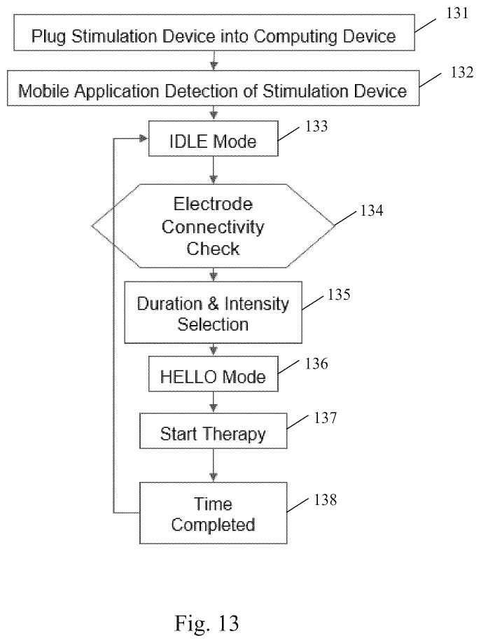

FIG. 13 is a flowchart showing the stimulation device operation steps of the Peripheral Nerve Stimulation (PNS) apparatus, as shown in FIG. 1, in accordance with one embodiment of the present invention. The flowchart in FIG. 13 illustrates the operational steps of a Peripheral Nerve Stimulation (PNS) device when used in conjunction with a smartphone. A detailed explanation of each block and the flow between them is described hereinafter:

At Step 131, Plug Stimulation Device into Computing Device: The first step involves connecting the stimulation device to a computing device, typically a smartphone, using a USB-C or Lightning port. This connection provides power and enables communication between the smartphone and the stimulation device.

At Step 132, Mobile Application Detection of Stimulation Device: Once connected, the mobile application detects the presence of the stimulation device. This is facilitated by the mobile application interfacing with the device through the established connection.

At Step 133, IDLE Mode: After detection, the stimulation device enters an IDLE mode. In this state, the device is powered on and ready but not yet delivering any stimulation. This mode allows the device to prepare for the next steps without applying any therapy to the user.

At Step 134, Electrode Connectivity Check: The system then performs an electrode connectivity check. The microprocessor interfaces with a connectivity/skin impedance check module to verify that the electrodes are properly attached to the user's skin. This ensures that the stimulation will be effective and safe. If the electrodes are not correctly positioned, the system prompts the user to adjust them.

At Step 135, Duration & Intensity Selection: Once electrode connectivity is confirmed, the user selects the duration and intensity of the treatment through the mobile application. Intensity levels can be set between 1 and 10, corresponding to an output intensity between 5 μA and 2 mA. The duration of treatment can be adjusted from 5 to 60 minutes. The device features an automatic shut-off after 60 minutes to prevent prolonged exposure.

At Step 136, HELLO Mode: After the user makes their selections, the device enters HELLO mode. This mode likely serves as a final confirmation and readiness state before starting the therapy. It may also include a brief self-check to ensure all systems are functioning correctly.

At Step 137, Start Therapy: The therapy begins according to the user's settings. The microprocessor controls the digital potentiometer to fine-tune the current and the waveform generator to produce the necessary electrical waveforms. These waveforms, with pulse widths ranging from 5 milliseconds to 1 second and frequencies of 0.5, 1.5, and 100 Hz, are transmitted through the electrodes to stimulate the nerves.

At Step 138, Time Completed: Once the selected treatment duration has elapsed, the device completes the therapy session. The integrated timer ensures that the device shuts off automatically after the set period, preventing any accidental over-stimulation and ensuring the safety of the user.

The described system integrates several safety and control features, including power management via a voltage regulator, connectivity checks, customizable stimulation settings, and automatic shut-off. These elements work together to provide a safe, effective, and user-friendly peripheral nerve stimulation therapy.

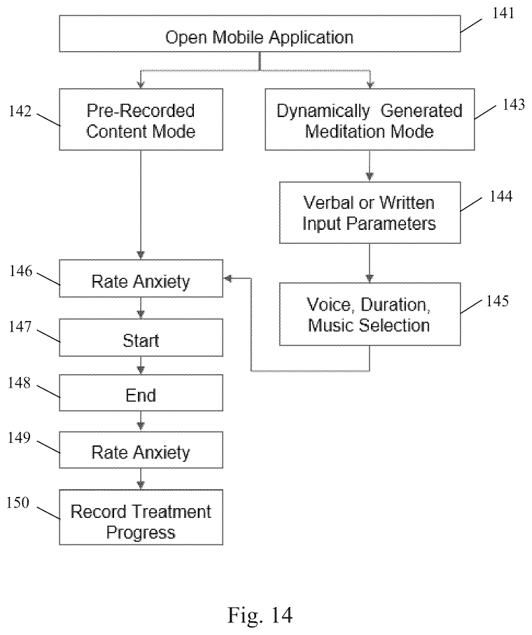

FIG. 14 is a flowchart showing the operational steps required to complete the interactive treatment sessions in the pre-recorded content mode or a dynamically generated meditation mode, in accordance with one embodiment of the present invention. The flowchart in FIG. 14 details the operational steps for completing interactive treatment sessions using a mobile application designed to manage anxiety and insomnia through a concealable, non-invasive peripheral nerve stimulation device. The method involves two main modes: pre-recorded content mode and dynamically generated meditation mode.

At Step 141, Open Mobile Application: The process begins when the user opens the mobile application designed for anxiety and insomnia treatment.

At Step 142, Pre-Recorded Content Mode: The user can select the pre-recorded content mode. In this mode, pre-recorded therapeutic content is used for the session.

At Step 143, Dynamically Generated Meditation Mode: Alternatively, the user can select the dynamically generated meditation mode. This mode generates personalized meditation sessions based on user input.

At Step 144, Verbal or Written Input Parameters: For dynamically generated meditation mode, the user provides input parameters, either verbally or in writing. These parameters include preferences for the session.

At Step 145, Voice, Duration, Music Selection: Based on the input parameters, the application configures the meditation session, selecting the appropriate voice, duration, and music to tailor the experience to the user's needs.

At Step 146, Rate Anxiety: Before starting any session, the user is prompted to rate their anxiety. This pre-session rating helps to establish a baseline for measuring the session's effectiveness. The anxiety rating is based on Generalized Anxiety Disorder (GAD) parameters, which include nervousness, control, excessive worry, tension, restlessness, irritability, and fear.

At Step 147, Start: The selected session, whether pre-recorded content or dynamically generated meditation, is initiated.

At Step 148, End: Upon completion of the session, playback or meditation is terminated.

At Step 149, Rate Anxiety: After the session, the user is prompted to rate their anxiety again. This post-session rating helps to assess the impact of the treatment.

At Step 150, Record Treatment Progress: The mobile application records the user's treatment progress, including pre- and post-session anxiety ratings. This record helps to track improvements and the effectiveness of the therapy over time.

The mobile application offers a method for treating anxiety and insomnia through two interactive treatment modes: pre-recorded content and dynamically generated meditation. The process involves opening the mobile application, selecting a mode, providing input parameters for dynamic sessions, rating anxiety before and after sessions, and recording treatment progress. The system ensures personalized and effective therapy by utilizing user feedback and preferences to tailor the treatment sessions.

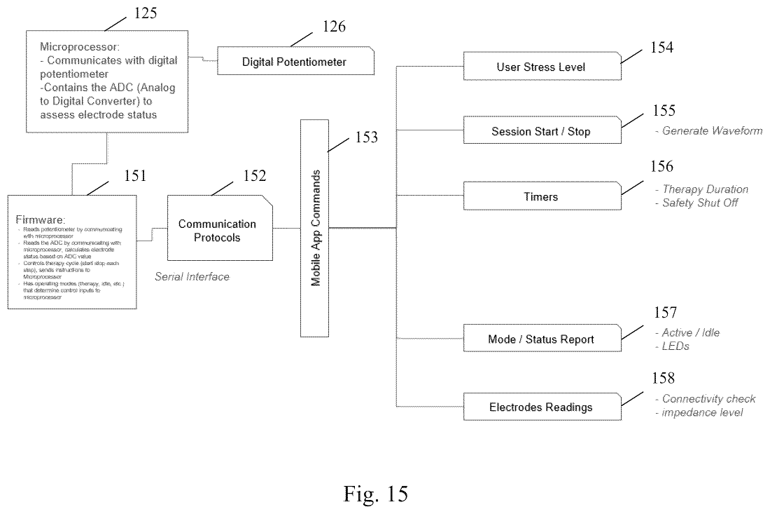

FIG. 15 is a flow diagram of the configuration of the stimulation device, in accordance with one embodiment of the present invention. This flow diagram explains how the system integrates hardware, firmware, and mobile application commands to deliver and manage therapy sessions. The microprocessor 125 serves as the central control unit of the stimulation device. This microprocessor 125 communicates with various components, including the digital potentiometer 126, and contains an ADC (Analog to Digital Converter) to assess electrode status. The microprocessor 125 controls the therapy cycle, reads the potentiometer values, and interprets ADC readings to ensure proper electrode connectivity and functionality. The digital potentiometer 126 fine-tunes the current based on inputs from the mobile application. This digital potentiometer 126 ensures that the electrical stimulation remains within safe and therapeutic limits. The digital potentiometer communicates its settings to the microprocessor for precise control of the stimulation intensity. The firmware 151 is responsible for reading the potentiometer by communicating with the microprocessor 125 and reading the ADC to assess electrode status. This firmware 151 calculates the electrode status based on ADC values and controls the therapy cycle (start/stop) by sending instructions to the microprocessor 125. The firmware also manages various operating modes, such as therapy and idle, determining the control inputs to the microprocessor 125. Further, the communication protocols 152 facilitate the serial communication between the mobile application and the microprocessor 125. They ensure that the commands from the mobile app are accurately transmitted and interpreted by the device's internal components.

The mobile application commands 153 includes sending various commands to the stimulation device, including user stress level, session start/stop, timers, mode/status report, and electrode readings. These commands help manage and monitor the therapy sessions. The user stress level 154 step includes the assessing by the mobile application and sending the user's stress level to the stimulation device. This input may be used to adjust the therapy parameters accordingly. The session Start/Stop 155 step includes the mobile application to command the stimulation device to start or stop the therapy session. When a session starts, the stimulation device generates the necessary waveform for nerve stimulation. In the timers 156 step, the mobile application sets the therapy duration and safety shut-off timers. These timers ensure the therapy does not exceed the safe operational period. In the Mode/Status Report 157 step, the mobile application can request or receive status updates from the stimulation device, indicating whether it is in active or idle mode. The LEDs on the device also reflect these statuses. In the Electrode Readings 158 step, the mobile application monitors the electrode connectivity check and impedance level to ensure the electrodes are properly attached and functioning.

The flow of information and control in this system starts with the mobile application, which sends commands to the device through communication protocols. The firmware interprets these commands and instructs the microprocessor 125 to perform various tasks, such as adjusting the digital potentiometer, checking electrode status, and controlling therapy sessions. The microprocessor 125, in turn, coordinates the overall operation, ensuring the therapy is delivered effectively and safely. This system integrates power management, safety checks, and customizable stimulation control, providing users with a reliable tool for managing peripheral nerve conditions. The seamless interaction between the mobile application, firmware, microprocessor, and other components ensures the therapy is both effective and safe.

While this invention has been described in terms of several preferred embodiments, there are alterations, permutations, and equivalents which fall within the scope of this invention. For example, although the peripheral nerve stimulation device has been primarily described in conjunction with a smartphone interface and specific communication protocols, it should be noted that some or all of the features described herein may be applied to other control interfaces and communication methodologies. It should also be noted that there are many alternative ways of implementing the methods and apparatuses of the present invention. Therefore, the following appended claims should be interpreted as including all such alterations, permutations, and equivalents as falling within the true spirit and scope of the present invention.

1. A non-invasive Peripheral Nerve Stimulation (PNS) system for treatment of anxiety and insomnia, said system comprising:
a headset including two ear hooks configured to ergonomically fit around ears of a user;
an adhesive electrode connected to each of the two ear hooks for placement on skin of mastoid processes of the user;
a stimulation device connected to the headset and configured to deliver a pulsed electrical current to each of the electrodes via a lead wire;
wherein the stimulation device is in communication with a computing device having a mobile application installed on the computing device, said mobile application is configured to control a plurality of output stimulation characteristics of the stimulation device, wherein the mobile application allows the user to adjust the output stimulation characteristics, monitor treatment progress, and access a plurality of interactive treatment sessions.

2. The PNS system of claim 1, wherein each of the ear hooks includes a snap port for attachment of the adhesive electrodes to the skin.

3. The PNS system of claim 1, wherein the electrodes are self-adhesive, conductive hydrogel disposable electrodes.

4. The PNS system of claim 1, wherein the output stimulation characteristics of the stimulation device include at least one of: intensity level of the pulsed electrical current, stimulation frequency, treatment duration, and operation mode.

5. The PNS system of claim 1, wherein the pulsed electrical current is delivered in a low-level pulsed electrical square wave form.

6. (canceled)

7. The PNS system of claim 1, wherein the mobile application is configured to measure a plurality of physiological biomarkers associated with anxiety intensity, wherein the physiological biomarkers are selected from heart rate variability, heart rate, skin impedance, skin conductance, blood oxygenation, and skin temperature, to provide personalized treatment recommendations.

8. The PNS system of claim 1, wherein the mobile application is configured to allow users to create a profile, respond to a validated psychological questionnaire, rate anxiety intensity before and after the interactive treatment sessions, track anxiety over time, and share data with healthcare providers.

9. (canceled)

10. The PNS system of claim 1, wherein the mobile application is configured to provide the plurality of interactive treatment sessions generated based on user inputs selected from music, narrator, language, session duration, and topic.

11. The PNS system of claim 1, wherein the stimulation device further comprises:
a microprocessor;
a digital potentiometer in communication with the microprocessor, the digital potentiometer configured to limit current surges, wherein the digital potentiometer includes a series of physical resistors to regulate current flow to a level not exceeding 2 mA;
a waveform generator connected to digital potentiometer and configured to generate the pulsed electrical current, wherein the microprocessor is configured to:
communicate with the digital potentiometer to read and adjust settings of the digital potentiometer;
communicate with an analog-to-digital converter (ADC) to read values of the ADC and check connectivity status of the electrode based on the ADC readings;
control the interactive treatment sessions based on user inputs; and
operate in different modes including a therapy mode and an idle mode based on the user adjusted output stimulation characteristics;
an integrated timer configured to shut-off the stimulation device after a predetermined time of continuous operation of the stimulation device.

12. The PNS system of claim 1, wherein the mobile application is available on multiple operating systems and requires users to create a password-protected account for secure access.

13. A method for treatment of anxiety and insomnia through a mobile application in communication with a concealable non-invasive peripheral nerve stimulation device, said method comprising:
presenting a user interface on the mobile application for selecting between two modes of a plurality of interactive treatment sessions including a pre-recorded content mode and a dynamically generated meditation mode;
in response to receiving a selection of the pre-recorded content mode:
prompting a user to input a pre-session anxiety rating;
initiating playback of the pre-recorded content;
terminating playback of the pre-recorded content;
prompting the user to input a post-session anxiety rating;
in response to receiving a selection of the dynamically generated meditation mode:
receiving verbal or written input parameters from the user;
configuring the dynamically generated meditation mode based on the input parameters, including selecting a voice, duration, and music;
prompting the user to input a pre-session anxiety rating;
initiating the dynamically generated meditation mode;
terminating the dynamically generated meditation mode;
prompting the user to input a post-session anxiety rating.

14. The method of claim 13, wherein the method further comprises recording treatment progress of the user on the user interface.

15. The method of claim 13, wherein the anxiety rating is based on Generalized Anxiety Disorder (GAD) parameters, wherein the GAD parameters includes at least one of nervousness, control, excessive worry, tension, restlessness, irritability and fear.

16. The method of claim 13, wherein the method further comprises, through the mobile application, creating a user profile by the user, responding to a validated psychological questionnaire, rating anxiety intensity before and after the interactive treatment sessions, tracking anxiety over time, and sharing data with healthcare providers.

17. (canceled)

18. A Peripheral Nerve Stimulation (PNS) apparatus for treatment of anxiety and insomnia, comprising:
a headset including two ear hooks configured to ergonomically fit around ears of a user;
an adhesive electrode connected to each of the two ear hooks for placement on skin of mastoid processes of the user; and
a stimulation device connected to the headset and configured to generate a plurality of output stimulation characteristics to deliver a pulsed electrical current to each of the electrodes via a lead wire, wherein the output stimulation characteristics are controllable by the user for the treatment of anxiety and insomnia.

19. The PNS apparatus of claim 18, wherein each of the ear hooks include snap ports for attachment of the adhesive electrodes to the skin.

20. The PNS apparatus of claim 18, wherein the electrodes are self-adhesive, conductive hydrogel disposable electrodes.

21. The PNS apparatus of claim 18, wherein the output stimulation characteristics of the stimulation device includes intensity level of the pulsed electrical current, stimulation frequency, treatment duration, and operation mode.

22. The PNS apparatus of claim 18, wherein the pulsed electrical current is delivered in a low-level pulsed electrical square wave form.

23. (canceled)

24. The PNS apparatus of claim 18, wherein the stimulation device further comprises:
a microprocessor;
a digital potentiometer in communication with the microprocessor, the digital potentiometer configured to limit current surges, wherein the digital potentiometer includes a series of physical resistors to regulate current flow to a level not exceeding 2 mA;
a waveform generator connected to digital potentiometer and configured to generate the pulsed electrical current, wherein the microprocessor is configured to:
communicate with the digital potentiometer to read and adjust settings of the digital potentiometer;
communicate with an analog-to-digital converter (ADC) to read values of the ADC and check connectivity status of the electrode based on the ADC readings; and
operate in different modes including a therapy mode and an idle mode based on the user controlled output stimulation characteristics;
an integrated timer configured to shut-off the stimulation device after a predetermined time of continuous operation of the stimulation device.
